(19) United States
(12) Patent Application Publication
(10) Pub. No.: US 2026/0245697 A1
(43) Pub. Date: Aug. 20, 2026

(54) Ambulatory Infusion Pump with Gradual Setting Adjustment Capabilities

(71) Applicant: Tandem Diabetes Care, Inc., San Diego, CA (US)

(72) Inventors: Nickolas Sherer, Escondido, CA (US); Thomas Ulrich, San Diego, CA (US); Ricardo Rueda, San Diego, CA (US); John Corbett, San Diego, CA (US)

(21) Appl. No.: 19/540,119

(22) Filed: Feb. 13, 2026

Related U.S. Application Data
(60) Provisional application No. 63/758,691, filed on Feb. 14, 2025.

(51) Int. Cl. G16H 20/17; A61B 5/00; A61M 5/172

(52) U.S. Cl. CPC G16H 20/17; A61B 5/4839; A61M 5/1723

(57) ABSTRACT

The present disclosure regards an ambulatory infusion pump with gradual setting adjustment capabilities. The pump includes a pump mechanism and a processor. The processor is configured to cause the pump mechanism to deliver the medicament at a flow rate determined based on a physiological parameter that has a first value. The processor is also configured to receive a request to modify the physiological parameter to have a second value. Additionally, the processor is configured to gradually change the physiological parameter from having the first value to having the second value while continuously determining the flow rate and causing the pump mechanism to deliver the medicament at the flow rate. Further, the processor is configured to determine the flow rate and cause the pump mechanism to deliver the medicament at the flow rate after changing the physiological parameter to the second value.

### RELATED APPLICATIONS

This application claims priority to U.S. Provisional Patent App. No. 63/758,691 (filed Feb. 14, 2025). The application also relates to U.S. Provisional App. Nos. 63/429,407 (filed Dec. 1, 2022), 63/615,481 (filed Dec. 28, 2023), and 63/615,579 (filed Dec. 28, 2023) and W.O. Patent App. No. PCT/US 2023/082084 (filed Dec. 1, 2023). Each of the foregoing applications is hereby incorporated in its entirety.

### TECHNICAL FIELD

The present disclosure relates, generally, to medical devices and, more specifically, to ambulatory infusion pumps for diabetes management.

### BACKGROUND

Ambulatory infusion pumps rely on a variety of physiological parameters during medicament delivery, such as basal rate and correction factor. These parameters can be used in tandem with additional data to determine delivery rates and bolus timing, and users can adjust the parameters to tailor their treatment to their current situation.

When adjusting physiological parameters, users expect their treatment to change in accordance with the newly adjusted parameters. An increased basal rate, for instance, should generally result in an increased medicament delivery rate. Nonetheless, immediate integration of parameter adjustment can result in unintended or unexpected changes to user treatment.

### SUMMARY

The present disclosure seeks to improve the handling of user adjustments to physiological parameters at an ambulatory infusion pump. These improvements are of particular utility when considered in conjunction with certain closed-loop algorithms for medicament delivery, such as the algorithms discussed in the applications listed in Related Applications, above.

In diabetes-treatment settings, medicament delivery algorithms calculate a medicament flow rate as a function of certain physiological parameters, as well as of glucose levels and insulin delivery history (and/or any other sensors or user inputs). Some of the parameters used in flow-rate calculations may undergo sudden changes (e.g., based on discreet user input), which might lead to unintended shifts in a closed-loop algorithm because such algorithms tend to respond more quickly to parameter adjustments than traditional administration algorithms. In such an instance, the user may receive a bolus of medicament in response to a parameter adjustment whereas a user employing a traditional algorithm would not receive a bolus. Accordingly, the present disclosure describes various examples of gradually accepting certain adjustments to physiological parameters in order to better meet user expectations (e.g., based on past experience with open-loop algorithms).

Exemplary embodiments include the following:

An Ambulatory Infusion Pump. The ambulatory infusion pump includes a memory, a pump mechanism, and a processor. The memory stores a physiological parameter of a user. The pump mechanism is configured to deliver a medicament to the user. The processor is configured to determine a flow rate for delivering the medicament to the user based on the physiological parameter having a first value. The processor is also configured to cause the pump mechanism to deliver the medicament to the user at the flow rate. Additionally, the processor is configured to receive a request to modify the physiological parameter from having the first value to having a second value different from the first value. Further, the processor is configured to gradually change the physiological parameter from having the first value to having the second value while continuously determining the flow rate based on the physiological parameter and continuously causing the pump mechanism to deliver the medicament to the user at the flow rate. Moreover, the processor is configured to, after changing the physiological parameter from having the first value to having the second value, determine the flow rate based on the physiological parameter and cause the pump mechanism to deliver the medicament to the user at the flow rate.

A Non-Transitory, Computer-Readable Medium. The computer-readable medium stores instructions that, when executed by a processor of an electronic device, cause the electronic device to perform operations. The operations include determining a flow rate for delivering a medicament to a user based on a physiological parameter of the user being stored in a memory and having a first value. The operations also include causing a pump mechanism to deliver the medicament to the user at the flow rate. Additionally, the operations include receiving a request to modify the physiological parameter from having the first value to having a second value different from the first value. Further, the operations include gradually changing the physiological parameter from having the first value to having the second value while continuously determining the flow rate based on the physiological parameter and continuously causing the pump mechanism to deliver the medicament to the user at the flow rate. Moreover, the operations include, after changing the physiological parameter from having the first value to having the second value, determining the flow rate based on the physiological parameter and causing the pump mechanism to deliver the medicament to the user at the flow rate.

A Computer-Implemented Method. The computer-implemented method is a method for gradually managing physiological parameter changes at an ambulatory infusion pump. The method includes determining a flow rate for delivering a medicament to a user based on a physiological parameter of the user being stored in a memory and having a first value. The method also includes causing a pump mechanism to deliver the medicament to the user at the flow rate. Additionally, the method includes receiving a request to modify the physiological parameter from having the first value to having a second value different from the first value. Further, the method includes gradually changing the physiological parameter from having the first value to having the second value while continuously determining the flow rate based on the physiological parameter and continuously causing the pump mechanism to deliver the medicament to the user at the flow rate. Moreover, the method includes, after changing the physiological parameter from having the first value to having the second value, determining the flow rate based on the physiological parameter and causing the pump mechanism to deliver the medicament to the user at the flow rate.

Based on the following Detailed Description, other configurations of the subject technology will be apparent to those skilled in the art. The Detailed Description describes various configurations of the subject technology, particularly with respect to illustrations thereof. Notwithstanding, the subject technology is capable of other and different configurations, and its several details are capable of modification in various other respects—all without departing from the scope of the subject technology. The Drawings and Detailed Description are therefore presented as illustrative in nature and should not be construed as restricting the present disclosure.

### BRIEF DESCRIPTION OF THE DRAWINGS

For a better understanding of the present disclosure, reference should be made to the Detailed Description, below, in conjunction with the following drawings. Like reference numerals refer to corresponding parts throughout the figures and the description.

FIGS. 1A and 1B are system diagrams that illustrate example ambulatory infusion systems for medicament delivery, according to various aspects of the subject technology.

FIGS. 2A-2C illustrate example approaches in accepting changes to a physiological parameter for an ambulatory infusion pump, according to various aspects of the subject technology.

FIG. 3 illustrates an example process for gradually managing physiological parameter changes at an ambulatory infusion pump, according to various aspects of the subject technology.

### DETAILED DESCRIPTION

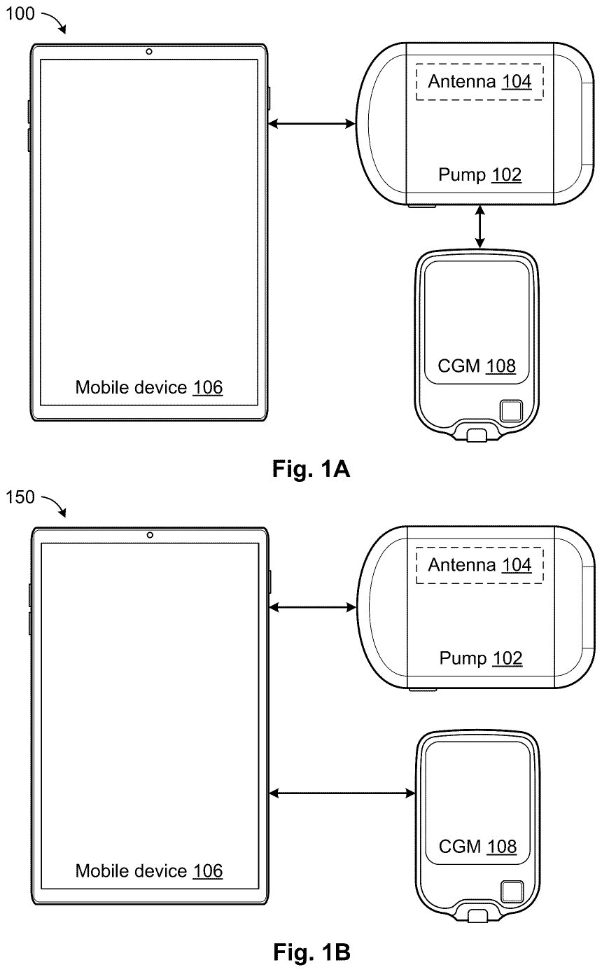

FIGS. 1A and 1B illustrate example ambulatory infusion systems 100 and 150 for medicament delivery, according to various aspects of the subject technology. Each system 100 and 150 includes an ambulatory infusion pump 102, a mobile device 106, and a continuous glucose monitor (CGM) 108.

In both systems 100 and 150, the pump 102 is configured to deliver a medicament to a user based at least in part on data or instructions received via its wireless antenna 104. For example, the pump 102 can receive BGL data and then determine a flow rate for medicament delivery based on the data. Or, as another example, the pump 102 can receive a bolus request and administer a bolus in accordance therewith. In some embodiments, the pump 102 is configured to store and deliver insulin to the user for BGL management; though, it can also be configured to deliver glucagon or another medicament for BGL management.

The mobile device 106 is configured to communicate with the pump 102. This communication may include the mobile device 106 sending instructions (e.g., a bolus request) or physiological parameters (or adjustments thereto) to the pump 102. The communication can also include the pump 102 sending notifications or status information back to the mobile device 106, which the mobile device 106 can then display for the user. In some embodiments, the mobile device 106 is a smartphone, a tablet, a smartwatch, or a laptop computer.

The CGM 108 is configured to collect data regarding the user's BGL and transmit the data to the pump 102, the mobile device 106, or a server in communication with at least one of the devices 102 or 106. In some embodiments, the CGM 108 collects the BGL data at regular intervals (e.g., every five minutes). Additionally, in some embodiments, the CGM 108 also transmits additional information regarding the BGL data to one of the aforesaid devices, such as a metric indicative of noise in the data.

In the first system 100, the pump 102 is in communication with both the mobile device 106 and the CGM 108. In this system 100, the pump 102 is configured to execute an algorithm for closed-loop operation based on BGL data from the CGM 108 and physiological parameters from the mobile device 106. By contrast, in the second system 150, the pump 102 is in communication only with the mobile device 106, which itself communicates with the CGM 108. In this system, either the pump 102 or the mobile device 106 may run the algorithm for medicament delivery. If the mobile device 106 manages the algorithm, it will send instructions (e.g., flow rate, bolus parameters) to the pump 102 for execution.

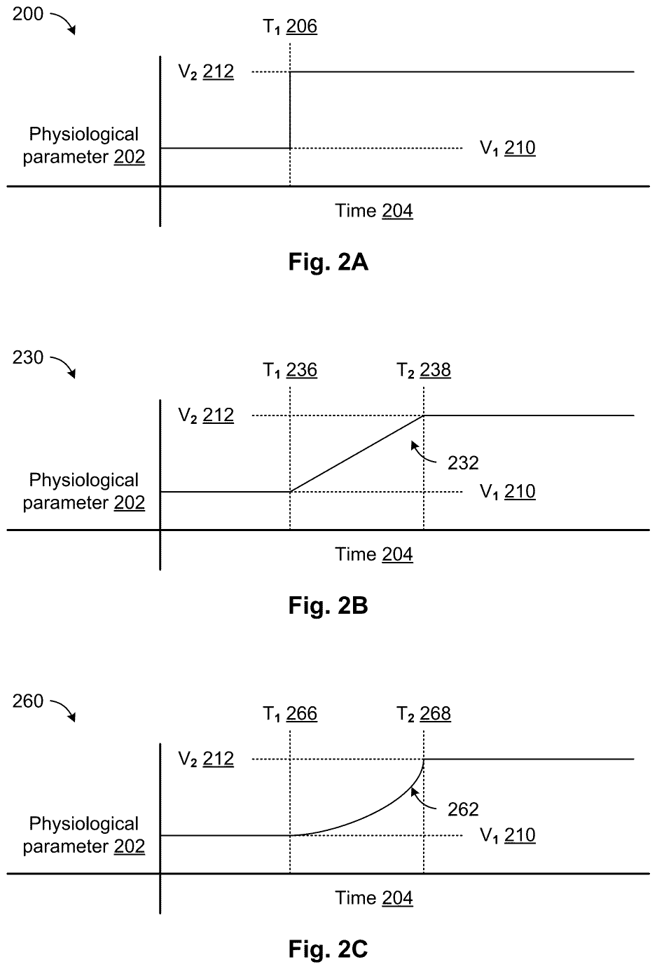

FIGS. 2A-2C illustrate example approaches to accepting changes to a physiological parameter for an ambulatory infusion pump (e.g., pump 102), according to various aspects of the subject technology. Each of the graphs 200, 230, and 260 includes a curve for the physiological parameter 202 (e.g., basal rate, correction factor) and axes for time 204 and magnitude (e.g., in units per hour, in units per milligram per deciliter).

In each of the graphs, the physiological parameter is changed from a first value (V₁) 210 to a second value (V₂) 212. These changes can be user initiated—for instance, the user may manually adjust their basal rate or correction factor in order to fine-tune their therapy. The changes can also be driven by user profiles: Many users set up profiles where their settings vary to account for changes to their physiological parameters due, for instance, to exercise, sleep, or eating. Additionally, the changes may be due to the user setting up a temporary parameter value, such as temporary a basal rate adjustment to account for a recently eaten meal or a temporary correction factor adjustment to account for ongoing exercise.

Under certain circumstances, the user may not expect the pump to make an immediate adjustment in response to a change to a physiological parameter. For instance, an ambulatory infusion pump operating in open loop mode will not immediately administer a bolus of insulin in response to a basal rate change. The same is true for pumps operating according to certain closed-loop algorithms (e.g., Control-IQ). Accordingly, the user may expect similar behavior from their ambulatory infusion pump.

In order to achieve this behavior, the present disclosure teaches various approaches for gradually accepting changes to physiological parameters. For contrast, the first graph 200 of FIG. 2A illustrates the immediate acceptance of changes to a physiological parameter 202. At time T₁ 206, the parameter 202 is changed from having the first value 210 to having the second value 212.

However, in the second graph 230, a linear function 232 is used to gradually change the physiological parameter 202 from having the first value 210 to having the second value 212. This change occurs over a period of time, from a first time T₁ 236 to a second time T₂ 238. In some embodiments, the linear function 232 is used to manage a change in correction factor such that the change is less likely to cause the pump to administer a bolus in response thereto.

Similarly, in the third graph 260, an exponential function 262 is used to change the physiological parameter 202 from having the first value 210 to having the second value 212. Like the change in the second graph 230, this change also occurs between respective first and second times 266 and 268. In some embodiments, the exponential function 262 is used to manage a change in basal rate such that the change is less likely to cause the pump to administer a bolus in response thereto.

Whether the ambulatory infusion pump changes the physiological parameter immediately or gradually can be pre-programmed, for instance, based on the type of parameter being changed. In some embodiments, carb ratio is changed immediately, whereas correction factor and basal rate are changed gradually. The benefit of changing the correction factor and basal rate can include better mimicking user-expected behavior, for instance, for users used to working with pumps in open-loop mode or certain closed-loop modes (e.g., based on Control-IQ).

Further, the pump can be configured to determine which type of function to use when changing a physiological parameter. For instance, the pump can use a lookup table indexed by parameter type. Or the pump can determine which function to use based on a profile of the user. For instance, if the pump determines based on the user profile that the user is accustomed to using a pump in open-loop mode or in a closed-loop mode based on a particular closed-loop algorithm (e.g., Control-IQ), then the pump can select the function type based on how the user might expect the pump to respond to the requested parameter adjustment.

After determining which type of function to use when changing the physiological parameter, in some embodiments, the pump is also configured to select variables for the function. The pump can determine which variables to use, for instance, based on the aforenoted user profiles and/or current infusion parameters. For instance, if the user's BGL is above a particular threshold, then the pump may reduce the amount of time needed to change the physiological parameter (e.g., by increasing the slope of a linear function). The pump can also determine the variables based on the type of physiological parameter being changed. For example, in some embodiments, the pump will select the variables to ensure that changing a correction factor takes thirty minutes'time. As another example, the pump can select variables to ensure changing a basal rate takes an amount of time equal to two-half lives of the medicament (e.g., ten minutes for insulin).

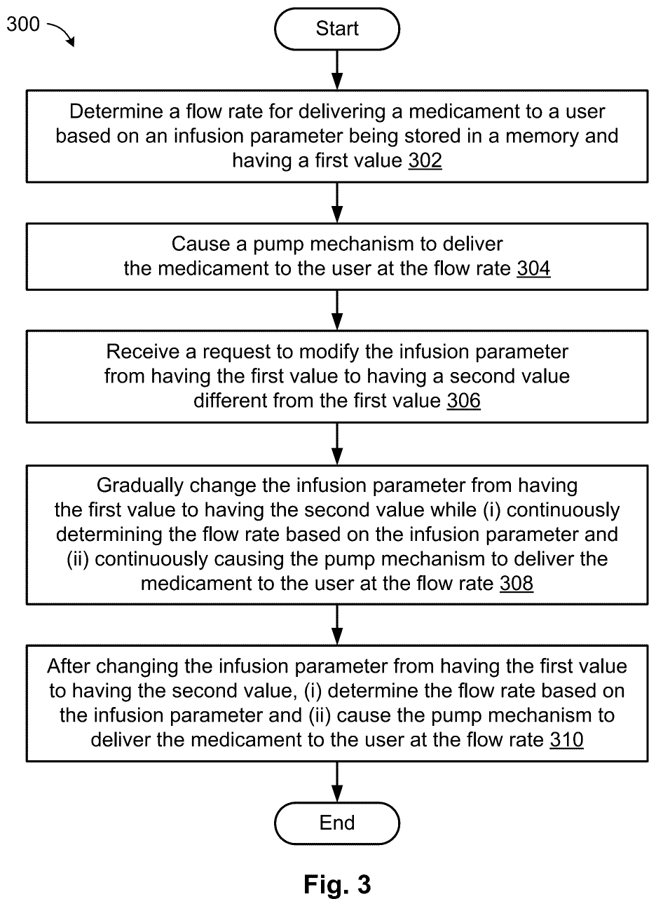

FIG. 3 illustrates an example process 300 for gradually managing physiological parameter changes at an ambulatory infusion pump, according to various aspects of the subject technology. The operations of the process 300 can be executed by devices discussed above with respect to FIGS. 1A, 1B, and 2A-5C, such as the ambulatory infusion pump 102 or mobile device 106. At least some of the operations can also be executed by a server connected to these devices 102 and 106.

In some embodiments, instructions stored in a non-transitory, computer-readable medium correspond to operations of the example process 300. In such embodiments, a processor of an electronic device (e.g., pump 102, mobile device 106) can execute the instructions to cause the electronic device to perform the corresponding operations.

The process 300 includes determining (302) a flow rate for delivering a medicament to a user based on a physiological parameter (e.g., a basal rate, a correction factor) being stored in a memory and having a first value (e.g., V₁ 210). The process 300 also includes causing (304) a pump mechanism to deliver the medicament to the user at the flow rate. Additionally, the process 300 includes receiving (306) a request to modify the physiological parameter from having the first value to having a second value (e.g., V₂ 212) different from the first value. For example, the request can be caused by manual user input or generated by an automatic shift from one user profile to another (e.g., a default profile to an exercise profile).

Further, the process 300 includes gradually changing (308) the physiological parameter from having the first value to having the second value (see, e.g., graphs 230 and 260) while continuously determining the flow rate based on the physiological parameter and continuously causing the pump mechanism to deliver the medicament to the user at the flow rate. For example, as discussed for FIGS. 2B and 2C, this can involve changing the physiological parameter using a linear function or an exponential function.

Moreover, the process 300 includes, after changing (308) the physiological parameter from having the first value to having the second value, determining (310) the flow rate based on the physiological parameter and causing (310) the pump mechanism to deliver the medicament to the user at the flow rate.

In some embodiments, the physiological parameter is a basal rate, and gradually changing the physiological parameter from having the first value to having the second value includes applying an exponential function (e.g., function 262) to the first value until a result of the exponential function is equal to the second value. In some of these embodiments, gradually changing the physiological parameter from having the first value to having the second value occurs over two half lives of the medicament.

Additionally, in some embodiments, the physiological parameter is a correction factor, and gradually changing the physiological parameter from having the first value to having the second value includes applying a linear function (e.g., function 232) to the first value until a result of the linear function is equal to the second value. In some of these embodiments, gradually changing the physiological parameter from having the first value to having the second value occurs over thirty minutes.

Further, in some embodiments, another physiological parameter is stored in a memory along with the first physiological parameter, and determining the flow rate for delivering the medicament to the user is further based on the other physiological parameter. In such embodiments, the process 300 further includes receiving a request to modify the other physiological parameter from having another first value to having another second value different from the other first value. Additionally, in such embodiments, the process 300 further includes gradually changing the other physiological parameter from having the other first value to having the other second value while continuously determining the flow rate based on the physiological parameter and the other physiological parameter and continuously causing the pump mechanism to deliver the medicament to the user at the flow rate. Further, in such embodiments, the process 300 further includes, after changing the other physiological parameter from having the other first value to having the other second value, determining the flow rate based on the physiological parameter and the other physiological parameter and causing the pump mechanism to deliver the medicament to the user at the flow rate.

Moreover, in some of these embodiments, the physiological parameter is a basal rate and the other physiological parameter is a correction factor. In such embodiments, gradually changing the physiological parameter from having the first value to having the second value includes applying an exponential function (e.g., function 262) to the first value until a result of the exponential function is equal to the second value, and gradually changing the other physiological parameter from having the other first value to having the other second value includes applying a linear function (e.g., function 232) to the first value until a result of the linear function is equal to the second value.

Furthermore, in some embodiments, a memory stores a first user profile (e.g., a default profile) associated with the first value for the physiological parameter (e.g., a default basal rate, a default correction factor) and a second user profile (e.g., an exercise profile) associated with the second value for the physiological parameter (e.g., an adjusted basal rate, an adjusted correction factor). In such embodiments, the process 300 further includes changing an active user profile from the first user profile to the second user profile based on yet another physiological parameter of the user (e.g., a heart rate, a BGL, a skin temperature, an oxygen saturation level, a ketone level, a temperature), and responsive to changing the active user profile, generating the request to modify the physiological parameter from having the first value to having the second value.

Additionally, in some embodiments, a wireless antenna (e.g., antenna 104) is configured to facilitate communication with a mobile device (e.g., mobile device 106), and the request to modify the physiological parameter includes a communication received from the mobile device via the wireless antenna, and the communication includes the second value and an indication of the physiological parameter.

Further, in some embodiments, the process 300 further includes receiving a carbohydrate ratio having another first value, where determining the flow rate for delivering the medicament to the user is not based on the carbohydrate ratio for the user. In such embodiments, the process 300 further includes receiving a request to modify the carbohydrate ratio from having the other first value to having another second value different from the first value and instantaneously changing the carbohydrate ratio from having the other first value to having the other second value (see, e.g., graph 200).

Illustrative Clauses. For further reference, example aspects of the present disclosure are included below as numbered clauses. These clauses are provided for illustrative purposes and are not intended to limit the subject technology.

Clause 1. An ambulatory infusion pump comprising: a memory storing a physiological parameter of a user; a pump mechanism configured to deliver a medicament to the user; and a processor configured to: determine a flow rate for delivering the medicament to the user based on the physiological parameter having a first value; cause the pump mechanism to deliver the medicament to the user at the flow rate; receive a request to modify the physiological parameter from having the first value to having a second value different from the first value; gradually change the physiological parameter from having the first value to having the second value while (i) continuously determining the flow rate based on the physiological parameter and (ii) continuously causing the pump mechanism to deliver the medicament to the user at the flow rate; after changing the physiological parameter from having the first value to having the second value, (i) determine the flow rate based on the physiological parameter and (ii) cause the pump mechanism to deliver the medicament to the user at the flow rate.

Clause 2. The ambulatory infusion pump of clause 1, wherein: the physiological parameter is a basal rate; and gradually changing the physiological parameter from having the first value to having the second value comprises applying an exponential function to the first value until a result of the exponential function is equal to the second value.

Clause 3. The ambulatory infusion pump of clause 2, wherein gradually changing the physiological parameter from having the first value to having the second value occurs over two half lives of the medicament.

Clause 4. The ambulatory infusion pump of any one of clauses 1-3, wherein: the physiological parameter is a correction factor; and gradually changing the physiological parameter from having the first value to having the second value comprises applying a linear function to the first value until a result of the linear function is equal to the second value.

Clause 5. The ambulatory infusion pump of clause 4, wherein gradually changing the physiological parameter from having the first value to having the second value occurs over thirty minutes.

Clause 6. The ambulatory infusion pump of any one of clauses 1-5, wherein: the memory further stores another physiological parameter, wherein determining the flow rate for delivering the medicament to the user is further based on the other physiological parameter; and the processor is further configured to: receive a request to modify the other physiological parameter from having another first value to having another second value different from the other first value; gradually change the other physiological parameter from having the other first value to having the other second value while (i) continuously determining the flow rate based on the physiological parameter and the other physiological parameter and (ii) continuously causing the pump mechanism to deliver the medicament to the user at the flow rate; after changing the other physiological parameter from having the other first value to having the other second value, (i) determine the flow rate based on the physiological parameter and the other physiological parameter and (ii) cause the pump mechanism to deliver the medicament to the user at the flow rate.

Clause 7. The ambulatory infusion pump of clause 6, wherein: the physiological parameter is a basal rate and the other physiological parameter is a correction factor; gradually changing the physiological parameter from having the first value to having the second value comprises applying an exponential function to the first value until a result of the exponential function is equal to the second value; and gradually changing the other physiological parameter from having the other first value to having the other second value comprises applying a linear function to the first value until a result of the linear function is equal to the second value.

Clause 8. The ambulatory infusion pump of any one of clauses 1-7, wherein: the memory further stores (i) a first user profile associated with the first value for the physiological parameter and (ii) a second user profile associated with the second value for the physiological parameter; and the processor is further configured to: change an active user profile from the first user profile to the second user profile based on yet another physiological parameter of the user; and responsive to changing the active user profile, generate the request to modify the physiological parameter from having the first value to having the second value.

Clause 9. The ambulatory infusion pump of any one of clauses 1-8, further comprising: a wireless antenna configured to facilitate communication with a mobile device; wherein the request to modify the physiological parameter comprises a communication received from the mobile device via the wireless antenna, and the communication comprises the second value and an indication of the physiological parameter.

Clause 10. The ambulatory infusion pump of any one of clauses 1-9, wherein the processor is further configured to: receive a carbohydrate ratio having another first value, wherein determining the flow rate for delivering the medicament to the user is not based on the carbohydrate ratio for the user; receive a request to modify the carbohydrate ratio from having the other first value to having another second value different from the first value; instantaneously change the carbohydrate ratio from having the other first value to having the other second value.

Clause 11. A non-transitory, computer-readable medium storing instructions that, when executed by a processor of an electronic device, cause the electronic device to perform operations comprising: determining a flow rate for delivering a medicament to a user based on a physiological parameter of the user being stored in a memory and having a first value; causing a pump mechanism to deliver the medicament to the user at the flow rate; receiving a request to modify the physiological parameter from having the first value to having a second value different from the first value; gradually changing the physiological parameter from having the first value to having the second value while (i) continuously determining the flow rate based on the physiological parameter and (ii) continuously causing the pump mechanism to deliver the medicament to the user at the flow rate; after changing the physiological parameter from having the first value to having the second value, (i) determining the flow rate based on the physiological parameter and (ii) causing the pump mechanism to deliver the medicament to the user at the flow rate.

Clause 12. The non-transitory, computer-readable medium of clause 11, wherein: the physiological parameter is a basal rate; and gradually changing the physiological parameter from having the first value to having the second value comprises applying an exponential function to the first value until a result of the exponential function is equal to the second value.

Clause 13. The non-transitory, computer-readable medium of clause 12, wherein gradually changing the physiological parameter from having the first value to having the second value occurs over two half lives of the medicament.

Clause 14. The non-transitory, computer-readable medium of any one of clauses 11-13, wherein: the physiological parameter is a correction factor; and gradually changing the physiological parameter from having the first value to having the second value comprises applying a linear function to the first value until a result of the linear function is equal to the second value.

Clause 15. The non-transitory, computer-readable medium of clause 14, wherein gradually changing the physiological parameter from having the first value to having the second value occurs over thirty minutes.

Clause 16. The non-transitory, computer-readable medium of any one of clauses 11-15, wherein: the memory further stores another physiological parameter, wherein determining the flow rate for delivering the medicament to the user is further based on the other physiological parameter; and the operations further comprise: receiving a request to modify the other physiological parameter from having another first value to having another second value different from the other first value; gradually changing the other physiological parameter from having the other first value to having the other second value while (i) continuously determining the flow rate based on the physiological parameter and the other physiological parameter and (ii) continuously causing the pump mechanism to deliver the medicament to the user at the flow rate; after changing the other physiological parameter from having the other first value to having the other second value, (i) determining the flow rate based on the physiological parameter and the other physiological parameter and (ii) causing the pump mechanism to deliver the medicament to the user at the flow rate.

Clause 17. The non-transitory, computer-readable medium of clause 16, wherein: the physiological parameter is a basal rate and the other physiological parameter is a correction factor; gradually changing the physiological parameter from having the first value to having the second value comprises applying an exponential function to the first value until a result of the exponential function is equal to the second value; and gradually changing the other physiological parameter from having the other first value to having the other second value comprises applying a linear function to the first value until a result of the linear function is equal to the second value.

Clause 18. The non-transitory, computer-readable medium of any one of clauses 11-17, wherein: the memory further stores (i) a first user profile associated with the first value for the physiological parameter and (ii) a second user profile associated with the second value for the physiological parameter; and the operations further comprise: changing an active user profile from the first user profile to the second user profile based on yet another physiological parameter of the user; and responsive to changing the active user profile, generating the request to modify the physiological parameter from having the first value to having the second value.

Clause 19. The non-transitory, computer-readable medium of any one of clauses 11-18, wherein the request to modify the physiological parameter comprises a communication received from a mobile device via a wireless antenna, and the communication comprises the second value and an indication of the physiological parameter.

Clause 20. A computer-implemented method for gradually managing physiological parameter changes at an ambulatory infusion pump, the method comprising: determining a flow rate for delivering a medicament to a user based on a physiological parameter of the user being stored in a memory and having a first value; causing a pump mechanism to deliver the medicament to the user at the flow rate; receiving a request to modify the physiological parameter from having the first value to having a second value different from the first value; gradually changing the physiological parameter from having the first value to having the second value while (i) continuously determining the flow rate based on the physiological parameter and (ii) continuously causing the pump mechanism to deliver the medicament to the user at the flow rate; after changing the physiological parameter from having the first value to having the second value, (i) determining the flow rate based on the physiological parameter and (ii) causing the pump mechanism to deliver the medicament to the user at the flow rate.

Further Consideration. The specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the invention described herein.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” and “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be appreciated that a message may be composed, transmitted, stored, received, and so on in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some embodiments, an n-input switch may be included to provide selective functionality where n is the number of inputs used to make the selection.

As used herein, the terms “correspond” or “corresponding” encompasses a structural, functional, quantitative, or qualitative correlation or relationship between two or more objects, data sets, information and the like, preferably where the correspondence or relationship may be used to translate one or more of the two or more objects, data sets, information and the like so to appear to be the same or equal. Correspondence may be assessed using one or more of a threshold, a value range, fuzzy logic, pattern matching, a machine-learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. An ambulatory infusion pump comprising:
a memory storing a physiological parameter of a user;
a pump mechanism configured to deliver a medicament to the user; and
a processor configured to:
determine a flow rate for delivering the medicament to the user based on the physiological parameter having a first value;
cause the pump mechanism to deliver the medicament to the user at the flow rate;
receive a request to modify the physiological parameter from having the first value to having a second value different from the first value;
gradually change the physiological parameter from having the first value to having the second value while (i) continuously determining the flow rate based on the physiological parameter and (ii) continuously causing the pump mechanism to deliver the medicament to the user at the flow rate;
after changing the physiological parameter from having the first value to having the second value, (i) determine the flow rate based on the physiological parameter and (ii) cause the pump mechanism to deliver the medicament to the user at the flow rate.

2. The ambulatory infusion pump of claim 1, wherein:
the physiological parameter is a basal rate; and
gradually changing the physiological parameter from having the first value to having the second value comprises applying an exponential function to the first value until a result of the exponential function is equal to the second value.

3. The ambulatory infusion pump of claim 2, wherein gradually changing the physiological parameter from having the first value to having the second value occurs over two half lives of the medicament.

4. The ambulatory infusion pump of claim 1, wherein:
the physiological parameter is a correction factor; and
gradually changing the physiological parameter from having the first value to having the second value comprises applying a linear function to the first value until a result of the linear function is equal to the second value.

5. The ambulatory infusion pump of claim 4, wherein gradually changing the physiological parameter from having the first value to having the second value occurs over thirty minutes.

6. The ambulatory infusion pump of claim 1, wherein:
the memory further stores another physiological parameter, wherein determining the flow rate for delivering the medicament to the user is further based on the other physiological parameter; and
the processor is further configured to:
receive a request to modify the other physiological parameter from having another first value to having another second value different from the other first value;
gradually change the other physiological parameter from having the other first value to having the other second value while (i) continuously determining the flow rate based on the physiological parameter and the other physiological parameter and (ii) continuously causing the pump mechanism to deliver the medicament to the user at the flow rate;
after changing the other physiological parameter from having the other first value to having the other second value, (i) determine the flow rate based on the physiological parameter and the other physiological parameter and (ii) cause the pump mechanism to deliver the medicament to the user at the flow rate.

7. The ambulatory infusion pump of claim 6, wherein:
the physiological parameter is a basal rate and the other physiological parameter is a correction factor;
gradually changing the physiological parameter from having the first value to having the second value comprises applying an exponential function to the first value until a result of the exponential function is equal to the second value; and
gradually changing the other physiological parameter from having the other first value to having the other second value comprises applying a linear function to the first value until a result of the linear function is equal to the second value.

8. The ambulatory infusion pump of claim 1, wherein:
the memory further stores (i) a first user profile associated with the first value for the physiological parameter and (ii) a second user profile associated with the second value for the physiological parameter; and
the processor is further configured to:
change an active user profile from the first user profile to the second user profile based on yet another physiological parameter of the user; and
responsive to changing the active user profile, generate the request to modify the physiological parameter from having the first value to having the second value.

9. The ambulatory infusion pump of claim 1, further comprising:
a wireless antenna configured to facilitate communication with a mobile device;
wherein the request to modify the physiological parameter comprises a communication received from the mobile device via the wireless antenna, and the communication comprises the second value and an indication of the physiological parameter.

10. The ambulatory infusion pump of claim 1, wherein the processor is further configured to:
receive a carbohydrate ratio having another first value, wherein determining the flow rate for delivering the medicament to the user is not based on the carbohydrate ratio for the user;
receive a request to modify the carbohydrate ratio from having the other first value to having another second value different from the first value;
instantaneously change the carbohydrate ratio from having the other first value to having the other second value.

11. A non-transitory, computer-readable medium storing instructions that, when executed by a processor of an electronic device, cause the electronic device to perform operations comprising:
determining a flow rate for delivering a medicament to a user based on a physiological parameter of the user being stored in a memory and having a first value;
causing a pump mechanism to deliver the medicament to the user at the flow rate;
receiving a request to modify the physiological parameter from having the first value to having a second value different from the first value;
gradually changing the physiological parameter from having the first value to having the second value while (i) continuously determining the flow rate based on the physiological parameter and (ii) continuously causing the pump mechanism to deliver the medicament to the user at the flow rate;
after changing the physiological parameter from having the first value to having the second value, (i) determining the flow rate based on the physiological parameter and (ii) causing the pump mechanism to deliver the medicament to the user at the flow rate.

12. The non-transitory, computer-readable medium of claim 11, wherein:
the physiological parameter is a basal rate; and
gradually changing the physiological parameter from having the first value to having the second value comprises applying an exponential function to the first value until a result of the exponential function is equal to the second value.

13. The non-transitory, computer-readable medium of claim 12, wherein gradually changing the physiological parameter from having the first value to having the second value occurs over two half lives of the medicament.

14. The non-transitory, computer-readable medium of claim 11, wherein:
the physiological parameter is a correction factor; and
gradually changing the physiological parameter from having the first value to having the second value comprises applying a linear function to the first value until a result of the linear function is equal to the second value.

15. The non-transitory, computer-readable medium of claim 14, wherein gradually changing the physiological parameter from having the first value to having the second value occurs over thirty minutes.

16. The non-transitory, computer-readable medium of claim 11, wherein:
the memory further stores another physiological parameter, wherein determining the flow rate for delivering the medicament to the user is further based on the other physiological parameter; and
the operations further comprise:
receiving a request to modify the other physiological parameter from having another first value to having another second value different from the other first value;
gradually changing the other physiological parameter from having the other first value to having the other second value while (i) continuously determining the flow rate based on the physiological parameter and the other physiological parameter and (ii) continuously causing the pump mechanism to deliver the medicament to the user at the flow rate;
after changing the other physiological parameter from having the other first value to having the other second value, (i) determining the flow rate based on the physiological parameter and the other physiological parameter and (ii) causing the pump mechanism to deliver the medicament to the user at the flow rate.

17. The non-transitory, computer-readable medium of claim 16, wherein:
the physiological parameter is a basal rate and the other physiological parameter is a correction factor;
gradually changing the physiological parameter from having the first value to having the second value comprises applying an exponential function to the first value until a result of the exponential function is equal to the second value; and
gradually changing the other physiological parameter from having the other first value to having the other second value comprises applying a linear function to the first value until a result of the linear function is equal to the second value.

18. The non-transitory, computer-readable medium of claim 11, wherein:
the memory further stores (i) a first user profile associated with the first value for the physiological parameter and (ii) a second user profile associated with the second value for the physiological parameter; and
the operations further comprise:
changing an active user profile from the first user profile to the second user profile based on yet another physiological parameter of the user; and
responsive to changing the active user profile, generating the request to modify the physiological parameter from having the first value to having the second value.

19. The non-transitory, computer-readable medium of claim 11, wherein the request to modify the physiological parameter comprises a communication received from a mobile device via a wireless antenna, and the communication comprises the second value and an indication of the physiological parameter.

20. A computer-implemented method for gradually managing physiological parameter changes at an ambulatory infusion pump, the method comprising:
determining a flow rate for delivering a medicament to a user based on a physiological parameter of the user being stored in a memory and having a first value;
causing a pump mechanism to deliver the medicament to the user at the flow rate;
receiving a request to modify the physiological parameter from having the first value to having a second value different from the first value;
gradually changing the physiological parameter from having the first value to having the second value while (i) continuously determining the flow rate based on the physiological parameter and (ii) continuously causing the pump mechanism to deliver the medicament to the user at the flow rate;
after changing the physiological parameter from having the first value to having the second value, (i) determining the flow rate based on the physiological parameter and (ii) causing the pump mechanism to deliver the medicament to the user at the flow rate.
